(19) United States
(12) Patent Application Publication
(10) Pub. No.: US 2024/0115485 A1
(43) Pub. Date: Apr. 11, 2024

(54) NAIL COMPOSITION CONTAINING DISPERSION OF ACRYLIC POLYMER PARTICLES

(71) Applicant: L'OREAL, Paris (FR)

(72) Inventors: I-Fan HSIEH, Scotch Plains, NJ (US); Tsang-Min HUANG, Scotch Plains, NJ (US)

(21) Appl. No.: 18/481,588

(22) Filed: Oct. 5, 2023

Related U.S. Application Data
(60) Provisional application No. 63/378,423, filed on Oct. 5, 2022.

(30) Foreign Application Priority Data
Dec. 15, 2022 (FR) FR2213420

(51) Int. Cl. A61K 8/81; A61Q 3/02

(52) U.S. Cl. CPC A61K 8/8152; A61Q 3/02; A61K 2800/31; A61K 2800/88

(57) ABSTRACT

A nail composition including a base composition and a dispersion of acrylic polymer particles. The dispersion of acrylic polymer particles is a dispersion of C1-C4 alkyl (meth)acrylate polymer particles stabilized with a stabilizer in a hydrocarbon oil, where the stabilizer is selected from the group consisting of: isobornyl (meth)acrylate homopolymers and statistical copolymers of isobornyl (meth)acrylate and of C1-C4 alkyl (meth)acrylate. The nail composition does not comprise a UV-curable material.

### TECHNICAL FIELD

The present invention relates to nail compositions comprising a dispersion of acrylic polymer particles and a base composition.

### BACKGROUND OF THE INVENTION

Nail compositions can consist of a layer of basecoat for adhesion on the nails, one or more color coats to enhance the color, and a layer of top coat for shine. A nail composition set is thus a system that typically contains a combination of base coat, color coat and/or top coat layers. Overtime, nail compositions applied to nails lose their shine and begin to chip. These properties are exhibited anywhere from the same day of application of the nail composition to 2-20 days after. This then requires the user to remove the nail composition and reapply, which can be time consuming.

It would be desirable to possess a nail composition that has one or more of the following properties (preferably, all of the following properties): good wear, and good shine, without modifying the hardness of the nail composition.

### SUMMARY OF THE INVENTION

The present invention relates to a nail composition comprising a dispersion of acrylic polymer particles and a base composition.

The present invention also relates to a nail composition set comprising (1) a color coat composition; and (2) a top coat composition comprising a dispersion of acrylic polymer particles and a base composition.

The present invention also relates to a nail composition set comprising (1) a color coat composition comprising a dispersion of acrylic polymer particles and a base composition; and (2) a top coat composition.

The present invention also relates to a nail composition set comprising (1) a color coat composition comprising a dispersion of acrylic polymer particles and a base composition; and (2) a top color coat composition comprising a dispersion of acrylic polymer particles and a base composition.

The present invention further relates to methods for making up and/or protecting nails comprising applying to the nails a nail composition comprising a dispersion of acrylic polymer particles and a base composition.

The present invention further relates to methods for making up and/or protecting nails comprising applying to the nails a nail composition set comprising (1) a color coat composition; and (2) a top coat composition comprising a dispersion of acrylic polymer particles and a base composition.

The present invention further relates to methods for making up and/or protecting nails comprising applying to the nails a nail composition set comprising (1) a color coat composition comprising a dispersion of acrylic polymer particles and a base composition; and (2) a top coat composition.

The present invention further relates to methods for making up and/or protecting nails comprising applying to the nails a nail composition set comprising (1) a color coat composition comprising a dispersion of acrylic polymer particles and a base composition; and (2) a top color coat composition comprising a dispersion of acrylic polymer particles and a base composition.

The present invention also relates to methods for improving wear and shine of a nail composition comprising adding a dispersion of acrylic polymer particles and a base composition wherein the addition of the dispersion of acrylic polymer particles and the base composition results in a nail composition having improved wear and shine properties.

It is to be understood that both the foregoing general description and the following detailed description are exemplary and explanatory only, and are not restrictive of the invention.

### BRIEF DESCRIPTION OF THE FIGURES

FIG. 1. is a graph illustrating representative tensile test data from Example A.

### DETAILED DESCRIPTION

In the following description of the invention and the claims appended hereto, it is to be understood that the terms used have their ordinary and accustomed meanings in the art, unless otherwise specified.

“About” or “approximately” as used herein means within 10% of the indicated number (e.g. “about 10°%” means 9%-11% and “about 2%” means 1.8%-2.2%).

“A” or “an” as used herein means “at least one.”

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3,2-4, 1-4, etc.

“Adhesion” as used herein, refers to chemical and/or physical bonding between a coating and a substrate. Good adhesion between nail polish and nail surface should translate to good wear properties on consumers.

“Adhesive agent” or “adhesive” means a substance that improves chemical and/or physical bonding between a coating and a substrate. In this invention, the adhesive agent improves bonding between compositions and the nail surface or other compositions.

“Film former”, “film-forming polymer” or “film forming agent” or “co-film former” as used herein means a polymer or resin that leaves a film on the substrate to which it is applied, for example, after a solvent accompanying the film former has evaporated.

“Free” or “devoid” of as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of solvents” means that non-aqueous solvents are preferably omitted (that is 0% by weight), but can be present in the composition at an amount of less than about 0.25% by weight, typically less than about 0.1% by weight, typically less than about 0.05% by weight, based on the total weight of the composition.

“Water free”, “anhydrous” or “free of water” herein means that water is preferably omitted (that is 0% by weight), but can be present in the composition at an amount of less than about 0.25% by weight, typically less than about 0.1% by weight, typically less than about 0.05% by weight, based on the total weight of the composition.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to nails and evaluating the color of the composition after an extended period of time. For example, the color of a composition may be evaluated immediately following application to nails and these characteristics may then be re evaluated and compared after a certain amount of time. Further, these characteristics may be evaluated with respect to other compositions, such as commercially available compositions.

“Making up” as used herein means to provide decoration (for example, color) to the nail.

“Protecting” as used herein means to inhibit damage to the nail (for example, chipping) by providing a protective layer on the nail.

“Nails”, “fingernail or “toenail” refers to a human keratinous substrate on a finger or toe which can be treated (decorated) with a single or multiple nail cosmetic compositions.

“Nail treatment system” or“nail composition set” means multiple compositions applied on the surface of nails.

“Nail composition” or “lacquer” or “nail polish” or “nail enamel” or “nail coating” or “nail film” refers to nail enamel usable as a basecoat, color coat, top coat, clear coat and protective coat applied on nails separately and/or as a combined application of the above.

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The compositions and methods of the present invention can comprise, consist of, or consist essentially of the essential elements and limitations of the invention described herein, as well as any additional or optional ingredients, components, or limitations described herein or otherwise useful.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

Dispersion of Acrylic Polymer Particles

According to the present invention, nail compositions containing a dispersion of acrylic polymer particles are provided. According to the present invention, the dispersions of acrylic polymer particles are dispersions of C1-C4 alkyl (meth)acrylate polymer particles stabilized with stabilizers based on isobornyl (meth)acrylate polymer in a hydrocarbon-based oil. Certain dispersion of acrylic polymer particles has been previously disclosed in PCT patent application serial no. PCT/EP2014/07800, and US patent publication nos. US20170172883A1 and US20170172890A1, the entire contents of each are hereby incorporated by reference.

According to preferred embodiments, the polymer of the particles is a C1-C4 alkyl (meth)acrylate polymer. The C1-C4 alkyl (meth)acrylate monomers may be chosen from methyl (meth)acrylate, ethyl (meth)acrylate, n-propyl (meth)acrylate, isopropyl (meth)acrylate, n-butyl (meth)acrylate and tert-butyl (meth)acrylate. Preferably, the monomer is a C1-C4 alkyl acrylate monomer. Preferably, the polymer of the particles is a methyl acrylate and/or ethyl acrylate polymer.

The polymer of the particles may also comprise an ethylenically unsaturated acid monomer or the anhydride thereof, chosen preferably from ethylenically unsaturated acid monomers comprising at least one carboxylic, phosphoric or sulfonic acid function, such as, for example, crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and/or salts thereof. Preferably, the ethylenically unsaturated acid monomer is chosen from (meth)acrylic acid, maleic acid and maleic anhydride. Most preferably, the ethylenically unsaturated acid monomer is acrylic acid. The salts may preferably be chosen from salts of alkali metals, for example sodium or potassium; salts of alkaline-earth metals, to example calcium, magnesium or strontium; metal salts, for example zinc, aluminium, manganese or copper; ammonium salts of formula NH4+; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles may thus comprise or consist essentially of 80% to 100%, preferably 85% to 95%, or about 90% by weight of C1-C4 alkyl (meth)acrylate and of 0 to 20%, preferably 5% to 15% or about 10% by weight of ethylenically unsaturated acid monomer, relative to the total weight of the polymer.

According to preferred embodiments, the polymer consists essentially of a polymer of one or more C1-C4 alkyl (meth)acrylate monomers.

According to preferred embodiments, the polymer consists essentially of a copolymer of methyl and ethyl acrylate. In the copolymer, ethyl acrylate is 50-95% by weight and methyl acrylate is 5-50% by weight, including all ranges and subranges therebetween.

The polymer of the particles may be chosen from, for example: methyl acrylate homopolymers; ethyl acrylate homopolymers; methyl acrylate/ethyl acrylate copolymers; methyl acrylate/ethyl acrylate/acrylic acid copolymers; methyl acrylate/ethyl acrylate/maleic anhydride copolymers; methyl acrylate/acrylic acid copolymers; ethyl acrylate/acrylic acid copolymers; methyl acrylate/maleic anhydride copolymers; and ethyl acrylate/maleic anhydride copolymers.

Preferably, the polymer of the particles is a non-crosslinked polymer.

The polymer of the particles of the dispersion preferably has a number-average molecular weight ranging from about 2000 to about 10,000,000, preferably ranging from about 150,000 to 500,000, including all ranges and subranges therebetween.

The polymer of the particles are preferably present in the dispersion in a content ranging from about 21% to about 58.5% by weight, preferably ranging from about 36% to about 42% by weight, relative to the total weight of the dispersion, including all ranges and subranges therebetween.

The stabilizer is preferably an isobornyl (meth)acrylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4. Preferably, the weight ratio ranges from about 4.5 to about 19, including all ranges and subranges therebetween.

Preferably, the stabilizer is chosen from, for example: isobornyl acrylate homopolymers; statistical copolymers of isobornyl acrylate/methyl acrylate; statistical copolymers of isobornyl acrylate/methyl acrylate/ethyl acrylate: statistical copolymers of isobornyl methacrylate/methyl acrylate, in the weight ratio described previously.

The stabilizing polymer preferably has a number-average molecular weight ranging from about 10,000 to about 400,000, preferably ranging from about 20,000 to about 200,000, including all ranges and subranges therebetween.

Although not wishing to be bound by any particular theory, it is believed that the stabilizer is in contact with the surface of the polymer particles and thus makes it possible to stabilize these particles at the surface in order to keep these particles in dispersion in the non-aqueous medium of the dispersion.

Preferably, the combination of the stabilizer+polymer of the particles present in the dispersion comprises from about 1% to about 50%, preferably 20% to 40%, or about 30% by weight of polymerized isobornyl (meth)acrylate, and from about 50% to about 90%, preferably 60% to 80%, or about 70% by weight of polymerized C1-C4 alkyl (meth)acrylate, relative to the total weight of the combination of the stabilizer+polymer of the particles.

Preferably, the combination of the stabilizer+polymer of the particles present in the dispersion comprises from about 15% to about 30% by weight of polymerized isobornyl (meth)acrylate, and from about 70% to about 85% by weight of polymerized C1-C4 alkyl (meth)acrylate, relative to the total weight of the combination of the stabilizer+polymer of the particles.

The oily medium of the polymer dispersion comprises a hydrocarbon-based oil.

The hydrocarbon-based oil is an oil that is liquid at room temperature (25° C.).

The term “hydrocarbon-based oil” means an oil formed essentially from, or even consisting of, carbon and hydrogen atoms, and optionally oxygen and nitrogen atoms, and not containing any silicon or fluorine atoms. It may contain, for example, alcohol, ester, ether, carboxylic acid, amine and/or amide groups.

The hydrocarbon-based oil may be chosen from, for example: hydrocarbon-based oils containing from 8 to 14 carbon atoms, preferably: branched C8-C14 alkanes, for instance C8-C14 isoalkanes of petroleum origin (also known as isoparaffins), for instance isododecane (also known as 2,2,4,4,6-pentamethylheptane), isodecane and, for example, the oils sold under the trade name Isopar or Permethyl, linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in Examples 1 and 2 of patent application WO 2008/155 059 from the company Cognis, the disclosure of which is hereby incorporated by reference, and mixtures thereof, short-chain esters (containing from 3 to 8 carbon atoms in total) such as ethyl acetate, methyl acetate, propyl acetate or n-butyl acetate, hydrocarbon-based oils of plant origin such as triglycerides consisting of fatty acid esters of glycerol, the fatty acids of which may have chain lengths varying from C4 to C24, these chains possibly being linear or branched, and saturated or unsaturated; these oils are especially heptanoic or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, guinea oil, rye oil, safflower oil, candlenut oil, passion-flower oil and musk rose oil; shea butter; or else caprylic/capric acid triglycerides, for instance those sold by the company Stéarineries Dubois or those sold under the names Miglyol 810®. 812® and 818® by the company Dynamit Nobel, synthetic ethers containing from 10 to 40 carbon atoms; linear or branched hydrocarbons of mineral or synthetic origin, such as petroleum jelly, polydecenes, hydrogenated polyisobutene such as Parleam®, squalane and liquid paraffins, and mixtures thereof, synthetic esters such as oils of formula R1COOR2 in which R1 represents a linear or branched fatty acid residue containing from 1 to 40 carbon atoms and R2 represents an, in particular, branched hydrocarbon-based chain containing from 1 to 40 carbon atoms, on condition that R1+R2≥10, for instance purcellin oil (cetostearyl octanoate), isopropyl myristate, isopropyl palmitate, C12 to C15 alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalkyl heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters, fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

Preferably, the hydrocarbon-based oil is nonpolar (formed solely from carbon and hydrogen atoms).

The hydrocarbon-based oil is preferably chosen from hydrocarbon-based oils containing from 8 to 14 carbon atoms, in particular the nonpolar oils described previously. Preferably, the hydrocarbon-based oil is isododecane.

The polymer particles of the dispersion preferably have an average size, especially a number-average size, ranging from about 50 to about 500 nm, preferably ranging from about 75 to about 400 nm, and preferably ranging from about 100 to about 250 nm, including all ranges and subranges therebetween.

In general, the dispersion according to the invention may be prepared in the following manner, which is given as an example.

The polymerization may be performed in dispersion, i.e. by precipitation of the polymer during formation, with protection of the formed particles with a stabilizer. In a first step, the stabilizing polymer is prepared by mixing the constituent monomer(s) of the stabilizing polymer, with a radical initiator, in a solvent known as the synthesis solvent, and by polymerizing these monomers. In a second step, the constituent monomer(s) of the polymer of the particles are added to the stabilizing polymer formed and polymerization of these added monomers is performed in the presence of the radical initiator.

When the non-aqueous medium is a non-volatile hydrocarbon-based oil, the polymerization may be performed in a nonpolar organic solvent (synthesis solvent), followed by adding the non-volatile hydrocarbon-based oil (which should be miscible with the said synthesis solvent) and selectively distilling off the synthesis solvent. A synthesis solvent which is such that the monomers of the stabilizing polymer and the free-radical initiator are soluble therein, and the polymer particles obtained are insoluble therein, so that they precipitate therein during their formation, is thus chosen. In particular, the synthesis solvent may be chosen from alkanes such as heptane or cyclohexane.

When the non-aqueous medium is a volatile hydrocarbon-based oil, the polymerization may be performed directly in the oil, which thus also acts as synthesis solvent. The monomers should also be soluble therein, as should the free-radical initiator, and the polymer of the particles obtained should be insoluble therein.

The monomers are preferably present in the synthesis solvent, before polymerization, in a proportion of about 5 to about 20% by weight. The total amount of monomers may be present in the solvent before the start of the reaction, or part of the monomers may be added gradually as the polymerization reaction proceeds. The free-radical initiator is preferably azobisisobutyronitrile or tert-butyl peroxy-2-ethylhexanoate.

The polymerization may be performed at a temperature ranging from about 70 to about 110° C.

The polymer particles are surface-stabilized, when they are formed during the polymerization, by means of the stabilizer.

The stabilization may be performed by any known means, and in particular by direct addition of the stabilizer, during the polymerization. The stabilizer is preferably also present in the mixture before polymerization of the monomers of the polymer of the particles. However, it is also possible to add it continuously, especially when the monomers of the polymer of the particles are also added continuously.

From about 10% to about 30% by weight, preferably from about 15% to about 25% by weight of stabilizer may be used, relative to the total weight of monomers used (stabilizer+polymer of the particles).

The polymer particle dispersion preferably comprises from about 30% to about 65% by weight, preferably from about 40% to about 60% by weight of solids, relative to the total weight of the dispersion.

Preferably, the oily dispersion may comprise a plasticizer, for example, a plasticizer chosen from tri-n-butyl citrate, tripropylene glycol monomethyl ether (INCI name: PPG-3 methyl ether) and trimethyl pentaphenyl trisiloxane (sold under the name Dow Corning PH-1555 HR Cosmetic Fluid by the company Dow Corning). These plasticizers make it possible to improve the mechanical strength of the polymer film. The plasticizer, if present, may be present in the oily dispersion in an amount ranging from about 5% to about 50% by weight, relative to the total weight of the polymer of the particles.

According to preferred embodiments, the polymer of the particles is a C1-C4 alkyl (meth)acrylate polymer; the stabilizer is an isobornyl (meth)acrylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4. For these statistical stabilizing copolymers, the defined weight ratio makes it possible to obtain a polymer dispersion that is stable, especially after storage for seven days at room temperature (25′ C.).

The dispersions according to the invention consist of particles, which are generally spherical, of at least one surface-stabilized polymer, in a non-aqueous medium.

Preferably, the amount of the dispersion of acrylic polymer particles present in the nail compositions of the present invention ranges from about 0.01% to about 20% by weight based on total weight of the composition, preferably about 0.5% to about 10% by weight based on the total weight of the composition, preferably about 1% to about 3% by weight based on the total weight of the composition, including all ranges and subranges therebetween.

Base Composition for the Nail Composition

According to preferred embodiments, the base composition for the nail compositions of the present invention is a solvent based composition. Set forth below are examples of ingredients which can be found in the nail compositions of the present invention, although all of the identified ingredients need not be present.

Film Forming Polymer

According to preferred embodiments, the nail compositions of the present invention comprise at least one non-photocurable film forming polymer. “Film-forming polymer” refers to a non-photocurable polymer suitable for forming alone (i.e. in the absence of an auxiliary film-forming agent or an external stimulus for example such as UV rays), a film suitable for being isolated, particularly a continuous adherent film, on a substrate, particularly on nails. Preferably, the film forming polymer is selected from the group consisting of radical or polycondensate type synthetic polymers, polymers of natural origin, and mixtures thereof.

Specific examples of suitable film forming polymers include, but are not limited to, polysaccharide derivatives, such as cellulose or guar gum derivatives including nitrocellulose and/or a polysaccharide ester or alkylether such as a polysaccharide consisting of repeat units comprising at least two identical or different rings and having a degree of substitution per saccharide unit between 1.9 and 3, preferably between 2.2 and 2.9, and preferably between 2.4 and 2.8, such as cellulose esters (such as cellulose acetobutyrates or cellulose acetopropionates), cellulose alkylethers (such as ethylcelluloses), and ethylguars. Specific examples of suitable film forming polymers also include, but are not limited to, alkyd resins, silicone-organic polymer hybrid compounds, polyurethanes, polyvinylbutyrals, and ketone/aldehyde resins, resins from aldehyde condensation products, such as aryl sulfonamide formaldehyde resins such as toluene sulfonamide formaldehyde resin, aryl-sulfonamide epoxy resins or ethyl tosylamide resins.

Preferably, if present, the at least one film forming polymer is at least one alkyd resin. Preferably, the at least one alkyd resin is a polyester comprising hydrocarbon chains of fatty acids. Such resins are described in particular in the Kirk-Othmer Encyclopedia of Chemical Technology, 4th edition, volume 2, pages 53 to 63, the content of which is hereby incorporated by reference. Such resins can be obtained by polymerization of polyols and polyacids or their corresponding anhydride in the presence of fatty acids, where the fatty acids can be employed “as is” or in the form of fatty acid triglycerides or in the form of oils during the synthesis of the alkyd resin. Due to the presence of hydrocarbon chains of fatty acids in the alkyd resin, alkyd resins are commonly defined by their oil length. Accordingly, “oil length of an alkyd resin” is understood to mean the percentage by weight of hydrocarbon chains of fatty acids present in the alkyd resin.

Examples of suitable polyols which can be employed in the synthesis of alkyd resins include, but are not limited to, at least one of pentaerythritol, trimethylolpropane, trimethylolethane, neopentyl glycol, propylene glycol, ethylene glycol, 1,6-hexanediol, 1,4-butanediol, diethylene glycol and, in particular, glycerol.

Examples of suitable polyacid or anhydride which can be employed in the synthesis of alkyd resins include, but are not limited to, at least one of, isophthalic acid, terephthalic acid, trimellitic anhydride, maleic anhydride, adipic acid, fumaric acid, azelaic acid, sebacic acid and, in particular, phthalic anhydride.

Examples of suitable fatty acids which can be employed in the synthesis of alkyd resins include, but are not limited to, at least one of fatty acids corresponding to the formula R—COOH, in which R denotes a saturated or unsaturated hydrocarbon radical preferably having from 7 to 45 carbon atoms, preferably from 9 to 35 carbon atoms, preferably from 15 to 35 carbon atoms and preferably from 15 to 21 carbon atoms. Mention may be made of, for example, palmitic acid, stearic acid, oleic acid, ricinoleic acid, linoleic acid, linolenic acid and, in particular, capric acid.

Fatty acids are present in the majority of oils of natural origin, in particular in the form of triglycerides. The triglycerides of fatty acids are esters resulting from the reaction of the three alcohol functional groups of glycerol with fatty acids, it being possible for these fatty acids to be identical or different. Oils of natural origin can thus be used during the polymerization. They can be chosen from, for example, linseed oil, China wood oil, oiticica oil, soybean oil, sunflower oil, safflower oil, castor oil, coconut oil, olive oil, palm oil, rapeseed oil, peanut oil and tall oil.

Specific examples of acceptable alkyd resins include, but are not limited to, at least one of those sold under the names “Beckosol ODE 230 70E” by Dainippon Ink & Chem (phthalic anhydride/glycerol/glycidyl decanoate copolymer in ethyl acetate at 70%), “Necowel 581®” (50% in soybean oil), “Necowel 585®” (20% in sunflower oil), “Necowel 580®” (20% in sunflower oil), “Necowel 586 N®” (50% in soybean oil), “Necowel EP 1161®” (50% in soybean oil), “Necowel EP 1213®” (20% in oil), “Necowel EP 2009®” (32% in sunflower oil), “Necowel EP 2019®” (20% in oil), “Necowel EP 2275®” (35% in oil), “Necowel EP 2329®” (34% in oil), and “Necowel EP 3016®” (30% in oil) by Ashland or “Uradil XP 515 AZ®” (73% in tall oil) or “Uradil XP 516 AZ®” (63% in tall oil) by DSM Resins. Beckosol ODE 230 70E (phthalic anhydride/glycerol/glycidyl decanoate copolymer in ethyl acetate at 70%) is particularly preferred.

Preferably, if present, the at least one film forming polymer is present in the nail composition of the present invention in amounts of active material generally ranging from about 2% to about 60%, preferably from about 5% to about 50%, and more preferably from about 8% to about 45%, by weight, based on the total weight of the composition, including all ranges and subranges in between.

Solvent

According to preferred embodiments, nail compositions optionally further comprising at least one solvent are provided. Any solvent typically found in nail polish compositions can be used. Suitable solvents include, but are not limited to, organic solvents which are liquid at ambient temperature. Examples of suitable solvents include, but are not limited to, ketones such as methyl ethyl ketone, methyl isobutyl ketone, diisobutyl ketone, isophorone, cyclohexanone or acetone; alcohols, such as ethanol, isopropanol, diacetone alcohol, 2-butoxyethanol or cyclohexanol; glycols, such as ethylene glycol, propylene glycol, pentylene glycol or glycerol; propylene glycol ethers, such as propylene glycol monomethyl ether, propylene glycol monomethyl ether acetate or dipropylene glycol mono(n-butyl) ether; short-chain esters (having a total of 2 to 7 carbon atoms), such as ethyl acetate, methyl acetate, propyl acetate, n-butyl acetate or isopentyl acetate; alkanes, such as decane, heptane, dodecane or cyclohexane; and their mixtures. Most preferred are short-chain esters (having a total of from 2 to 8 carbon atoms).

Preferably, solvent is present in the nail composition of the present invention in amounts of active material generally ranging from about 0.1% to about 90%, preferably from about 5% to about 60%, and more preferably from about 15% to about 40%, by weight, based on the total weight of the composition, including all ranges and subranges in between.

In a preferred embodiment, the solvent is not water and the nail composition is water-free or anhydrous.

Colorant

According to preferred embodiments, nail compositions further comprising at least one colorant are provided. Any colorant typically found in nail polish compositions can be used. Suitable colorants include, but are not limited to, lipophilic dyes, pigments, pearlescent agents, glitter, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C: Red No. 10, 11, 12, and 13. D&C Red No. 7, D&C Red No. 5 and 6, and D&D Red No. 34, as well as lakes such as D&C Yellow Lake No. 5 and D&C Red Lake No. 2.

Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Preferably, if present, the at least one colorant is present in the nail composition of the present invention in amounts of active material generally ranging from about 0.1% to about 10%, preferably from about 0.25% to about 7%, and more preferably from about 0.5% to about 3.5%, by weight, based on the total weight of the nail composition, including all ranges and subranges in between Auxiliaries/Additives.

The nail compositions of the present invention may additionally comprise an additive or auxiliary commonly used in cosmetic compositions and known to a person skilled in the art as being capable of being incorporated into a nail polish or varnish composition. Such additives or auxiliaries may be chosen from plasticizers, thickeners, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils. UV screening agents, sunscreens, moisturizing agents, vitamins. proteins, ceramides, plant extracts, fibers, and the like, and their mixtures.

In a preferred embodiment, there are no UV curing agents in the nail composition. A UV-curing agent includes photocrosslinkable compounds and photoinitiators. The nail composition hardens over time without any UV curing agents that are cured with UV light. The nail composition hardens by solvent evaporation in an ambient atmosphere in less than 2 hours.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable. The composition may be in any galenic form normally employed in the cosmetic and dermatological fields which is suitable for topical administration onto nails.

Nail Composition Set

According to the present invention, a nail composition set comprising at least one color coat and at least one top coat are provided. The nail composition set of the present invention can optionally further comprise at least one primer coat and/or at least one base coat.

It should be understood that each coat or layer in the nail composition set, itself, can comprise one or more layers of each composition. Thus, the at least one primer can comprise one or more primer layers; the at least one basecoat can comprise one or more basecoat layers; the at least one color coat can comprise one or more color coat layers; and the at least one top coat can comprise one or more top coat layers. Preferably, each primer, basecoat, color coat and top coat contains three or fewer layers or compositions, more preferably two or fewer layers or compositions, and most preferably a single layer or composition.

According to the present invention, the nail composition set is a nail composition comprising a dispersion of acrylic polymer particles in accordance with the present invention. The other composition(s) of the nail composition set may be any suitable composition for application to nails. Both or one of a top coat and color coat in the nail composition set may comprise a dispersion of acrylic polymer particles.

The present invention also relates to methods for improving wear and shine of a nail composition comprising adding a dispersion of acrylic polymer particles and a base composition wherein the addition of the dispersion of acrylic polymer particles and the base composition results in a nail composition having improved wear and shine properties. The wear and shine properties are improved compared to a composition that does not include a dispersion of acrylic polymer particles.

“Shine” or “Gloss” in compositions as used herein refers to compositions having with an average gloss, measured at 20°, of greater than or equal to 35, for example 40, preferably 45, 55, 60, 65, 70, 75, 80, 85, or 90° including all ranges and subranges therebetween. “Shine enhancing agent” or “shine increasing agent” in accordance with the present invention means increasing shine or, as the case may be, mitigating or reducing any reduction in shine that may result from the use of corresponding amounts of non-shine enhancing materials.

Wear improvement provides less chipping of the nail composition after application to the nail. Components of the nail composition can improve the adhesion to the nail following application and provide a sufficient hardness to prevent chipping. The hardness may be from 30-60, preferably 35-55, or 40-50, including all ranges and subranges therebetween as measured with a Persoz hardness tester.

According to such methods, preferably, the amount of the dispersion of acrylic polymer particles added to the nail composition is generally from about 0.01% to about 20%, preferably from about 1% to about 15%, and more preferably from about 3% to about 10%, by weight, based on the total weight of the composition, including all ranges and subranges in between.

According to preferred embodiments of the present invention, methods of making up or protecting nails comprising applying to the nails a nail composition comprising a dispersion of acrylic polymer particles to nails in an amount sufficient to makeup or protect the nails are provided.

The compositions according to the invention can be manufactured by known processes used generally in the cosmetics or dermatological field.

Preferred embodiments of the present invention include but are not limited to:

A nail composition comprising a base composition; and a dispersion of acrylic polymer particles; wherein the dispersion of acrylic polymer particles is a dispersion of C1-C4 alkyl (meth)acrylate polymer particles stabilized with a stabilizer in a hydrocarbon oil,

wherein the stabilizer is selected from the group consisting of: isobornyl (meth)acrylate homopolymers; and statistical copolymers of isobornyl (meth)acrylate and of C1-C4 alkyl (meth)acrylate, and wherein the nail composition does not comprise a UV-curable material;

The nail composition of the preceding embodiment, comprising 1-40% by weight of the stabilizer based on the total weight of the stabilizer and acrylic polymer particles;

The nail composition of any preceding embodiment, comprising 0.01-20% by weight of the dispersion of acrylic polymer particles based on the total weight of the composition;

The nail composition of any preceding embodiment, wherein a polymer of the acrylic polymer particles is methyl acrylate and/or ethyl acrylate polymer;

The nail composition of any preceding embodiment, wherein the acrylic polymer particles comprise 50-95% by weight ethyl acrylate and 5-50% by weight methyl acrylate;

The nail composition of any preceding embodiment, wherein the acrylic polymer particles further comprise an ethylenically unsaturated acid monomer or an anhydride thereof;

The nail composition of any preceding embodiment, wherein the acrylic polymer particles further comprise an ethylenically unsaturated acid monomer selected from the group consisting of crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid, acrylamidoglycolic acid, and salts thereof;

The nail composition of any preceding embodiment, wherein the acrylic polymer particles comprise from 80% to 100% by weight of C1-C4 alkyl (meth)acrylate and from 0 to 20% by weight of an ethylenically unsaturated acid monomer, relative to the total weight of the acrylic polymer particles;

The nail composition of any preceding embodiment, wherein the acrylic polymer particles are at least one selected from the group consisting of: methyl acrylate homopolymers; ethyl acrylate homopolymers; methyl acrylate/ethyl acrylate copolymers; methyl acrylate/ethyl acrylate/acrylic acid copolymers; methyl acrylate/ethyl acrylate/maleic anhydride copolymers; methyl acrylate/acrylic acid copolymers; ethyl acrylate/acrylic acid copolymers; methyl acrylate/maleic anhydride copolymers; and ethyl acrylate/maleic anhydride copolymers;

The nail composition of any preceding embodiment, wherein the stabilizer is at least one selected from the group consisting of: isobornyl acrylate homopolymers; statistical copolymers of isobornyl acrylate/methyl acrylate; statistical copolymers of isobornyl acrylate/methyl acrylate/ethyl acrylate; and statistical copolymers of isobornyl methacrylate/methyl acrylate;

The nail composition of any preceding embodiment, having a shine from 60-90°;

The nail composition of any preceding embodiment, having a hardness from 30-90 measured with a Persoz hardness test;

The nail composition of any preceding embodiment, wherein the nail composition is anhydrous;

The nail composition of any preceding embodiment, wherein the nail composition is a top coat;

The nail composition of any preceding embodiment, wherein the nail composition is a color coat;

A nail composition set comprising the nail composition of any preceding embodiment; and

A kit comprising the nail composition of any preceding embodiment.

Unless otherwise indicated, all numbers expressing quantities of ingredients, reaction conditions, and so forth used in the specification and claims are to be understood as being modified in all instances by the tem “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example A—Measurement of the Nail Composition Properties

The gloss or shine is measured using a gloss meter, for example by spreading a layer of the composition to be tested, between 50 μm and 150 μm in thickness, on a white Leneta contrast card using an automatic spreader. The residual tacky layer is wiped off with lint free cotton saturated in alcohol solvent, and then the gloss is measured at 20° and 60° using a Byk Gardner gloss meter of reference microTRI-GLOSS. This measurement is repeated at least three times, and the average gloss in GU (gloss units) is the average of the at least three measurements carried out.

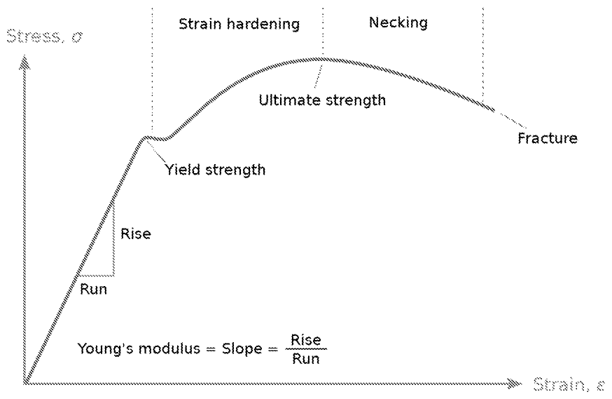

The elongation, break strength and young modulus are measured using Dynamic mechanical analysis (DMA) with tensile test method. Sample prepared by coating the formula on a Polypropylene (PP) plate with film thickness between 50 μm and 150 μm. The deposited film is dried under atmosphere for 24 hours. After dried, the test sample is cut into long strip and mount into the tensile grip for elongation test. Test is performed at 37° C. with the elongation rate at 300% per minutes. Representative tensile test data are shown in FIG. 1. The Young's modulus and elongation at break (Strain reading at fracture) are calculated from the graph as depicted in FIG. 1.

### Example B—Preparation of the Dispersion of Acrylic Polymer Particles

An example of the dispersion of acrylic polymer particles was made by the following method.

In a first step, 1300 g of isododecane, 337 g of isobornyl acrylate, 28 g of methyl acrylate and ethyl acrylate were placed in a reactor. The isobornyl acrylate/methyl acrylate mass ratio is. The mixture was heated at 90° C. under argon with stirring. After 2 hours of reaction, 1430 g of isododecane were added to the reactor feedstock and the mixture was heated to 90° C.

In a second step, a mixture of 1376 g of methyl acrylate, 1376 g of isododecane were run in over 2 hours 30 minutes, and the mixture was left to react for 7 hours, 3.3 litres of isododecane were then added and part of the isododecane was evaporated off to obtain a solids content of 50% by weight. A dispersion of methyl acrylate particles stabilized with a statistical copolymer stabilizer containing 92% isobornyl acrylate and 8% methyl acrylate in isododecane was obtained.

The dispersion contains in total (stabilizer+particles) 80% methyl acrylate and 20% isobornyl acrylate.

The polymer particles of the dispersion have a number-average size of about 160 nm.

The dispersion is stable after storage for 7 days at room temperature (25′ C.).

The polymer and oil weight ratio is 1:1 in this polymer particle dispersion.

The weight percentage of the components in the acrylic polymer particles is shown in Table 1.

TABLE 1
Acrylic polymer particles weight components

| Component | Monomer | Weight percent |
|---|---|---|
| Stabilizer | Isobornyl Acrylate | 20 |
| C1-C4 alkyl | Methyl Acrylate | 10 |
| (meth)acrylate | Ethyl Acrylate | 60 |
| polymer particles | | |
| Ethylenically | Acrylic Acid | 10 |
| unsaturated acid | | |
| monomer | | |

### Examples 1-5

The dispersion of acrylic polymer particles was mixed with a color coat base composition to form the nail composition. The dispersion was added directly to the base composition and mixed using overhead mixer Examples 1-5 are examples of different concentrations (0.2%, 0.5%, 1%, 3%, 5%) of the dispersion of acrylic polymer particles added to the color coat base composition to form the nail composition. The components of the color coat base composition are shown in Table 2.

TABLE 2
Base composition weight components

| Component | Weight percent |
|---|---|
| Solvent | 70 |
| Nitrocellulose | 10 |
| Tosylamide epoxy resin | 8 |
| Plasticizer | 9 |

Below is the top coat composition:

| Component | Weight percent |
|---|---|
| Solvent | 78 |
| Nitrocellulose | 10 |
| Other film former | 5-6 |
| Plasticizer | 9 |

The presence of the dispersion of acrylic polymer particles in the nail composition increases the shine while maintaining a hardness between 30-50 as show in Table 3. This is likely due to the strong mechanical properties of the C1-C4 alkyl (meth)acrylate polymer particles, in combination with the shine enhancing properties of the stabilizer and the improved adhesion properties of the ethylenically unsaturated acid monomer.

TABLE 3
Properties of Examples 1-5

| | Dispersion Concentration | | | | |
|---|---|---|---|---|---|
| | Ex. 1 | Ex. 2 | Ex. 3 | Ex. 4 | Ex. 5 |
| | 0.2% | 0.5% | 1% | 3% | 5% |
| Shine (20°) | 61.5 | 61.1 | 63.1 | 65 | 68.7 |
| Hardness | 35 | 45 | 38 | 49 | 32 |

A color coat was applied to a nail and allowed to dry and then the top coat with the added amount of dispersion of acrylic polymer particles was applied over the color coat and allowed to dry. The shine and wear of the color coat with the top coat with the dispersion of acrylic polymer particles was monitored over a 10 day period. A comparative example of the same color coat and top coat applied but without the dispersion of acrylic polymer particles was also monitored.

TABLE 4
Performance of Examples 1-5

| | Dispersion Concentration | | | | |
|---|---|---|---|---|---|
| | Ex. 1 | Ex. 2 | Ex. 3 | Ex. 4 | Ex. 5 |
| | 0.2% | 0.5% | 1% | 3% | 5% |
| Application | = | = | = | = | = |
| Wear at Day 7 | = | + | + | = | = |
| Shine at Day 7 | = | = | + | + | + |
| Wear at Day 10 | = | = | + | + | + |
| Shine at Day 10 | = | = | + | + | + |

Table 4 shows the performance of the nail compositions after 10 days relative to the comparative example. An “=” symbol indicates that the nail composition has similar performance to that of the comparative example. A “+” symbol indicates that the nail composition performs better than the comparative example. In the case of wear, better performance means less chipping, and in the case of shine better performance means a shine is maintained after 10 days.

Addition of 0.2% of the dispersion of acrylic polymer particles did not result in any differences from the comparative example, and 0.5% only improved wear up to 7 days. Examples 3-5 with of 1%, 3%, and 5% added dispersion of acrylic polymer particles improved the shine and wear of the nail composition for the 10 day duration. Greater than 5% of the dispersion of acrylic polymer particles in the composition causes incompatibility with the base composition. Less than 1% of the dispersion of acrylic polymer particles in the composition does not result in an improvement of shine, due to the low concentration.

1. A nail composition comprising:
a base composition; and
a dispersion of acrylic polymer particles;
wherein the dispersion of acrylic polymer particles is a dispersion of C1-C4 alkyl (meth)acrylate polymer particles stabilized with a stabilizer in a hydrocarbon oil,
wherein the stabilizer is selected from the group consisting of: isobornyl (meth)acrylate homopolymers; and statistical copolymers of isobornyl (meth)acrylate and of C1-C4 alkyl (meth)acrylate,
wherein the nail composition does not comprise a UV-curable material.

2. The nail composition of claim 1, comprising 1-40% by weight of the stabilizer based on the total weight of the stabilizer and acrylic polymer particles.

3. The nail composition of claim 1, comprising 0.01-20% by weight of the dispersion of acrylic polymer particles based on the total weight of the composition.

4. The nail composition of claim 1, wherein a polymer of the acrylic polymer particles is methyl acrylate and/or ethyl acrylate polymer.

5. The nail composition of claim 1, wherein the acrylic polymer particles comprise 50-95% by weight ethyl acrylate and 5-50% by weight methyl acrylate.

6. The nail composition of claim 1, wherein the acrylic polymer particles further comprise an ethylenically unsaturated acid monomer or an anhydride thereof.

7. The nail composition of claim 1, wherein the acrylic polymer particles further comprise an ethylenically unsaturated acid monomer selected from the group consisting of crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid, acrylamidoglycolic acid, and salts thereof.

8. The nail composition of claim 1, wherein the acrylic polymer particles comprise from 80% to 100% by weight of C1-C4 alkyl (meth)acrylate and from 0 to 20% by weight of an ethylenically unsaturated acid monomer, relative to the total weight of the acrylic polymer particles.

9. The nail composition of claim 1, wherein the acrylic polymer particles are at least one selected from the group consisting of: methyl acrylate homopolymers; ethyl acrylate homopolymers; methyl acrylate/ethyl acrylate copolymer; methyl acrylate/ethyl acrylate/acrylic acid copolymers; methyl acrylate/ethyl acrylate/maleic anhydride copolymers; methyl acrylate/acrylic acid copolymers; ethyl acrylate/acrylic acid copolymers; methyl acrylate/maleic anhydride copolymers; and ethyl acrylate/maleic anhydride copolymers.

10. The nail composition of claim 1, wherein the stabilizer is at least one selected from the group consisting of: isobornyl acrylate homopolymers; statistical copolymers of isobornyl acrylate/methyl acrylate; statistical copolymers of isobornyl acrylate/methyl acrylate/ethyl acrylate; and statistical copolymers of isobornyl methacrylate/methyl acrylate.

11. The nail composition of claim 1, having a shine from 60-90°.

12. The nail composition of claim 1, having a hardness from 30-90 measured with a Persoz hardness test.

13. The nail composition of claim 1, wherein the nail composition is anhydrous.

14. The nail composition of claim 1, wherein the nail composition is a top coat.

15. The nail composition of claim 1, wherein the nail composition is a color coat.

16. A nail composition set comprising the nail composition of claim 1.

17. A kit comprising the nail composition of claim 1.
